(19) United States
(12) Patent Application Publication
(10) Pub. No.: US 2018/0071452 A1
(43) Pub. Date: Mar. 15, 2018

(54) System and Method for Optimizing Contrast Imaging of a Patient

(71) Applicant: Siemens Healthcare GmbH, Erlangen (DE)

(72) Inventors: Puneet Sharma, Monmouth Junction, NJ (US); Lucian Mihai Itu, Brasov (RO)

(21) Appl. No.: 15/690,472

(22) Filed: Aug. 30, 2017

Related U.S. Application Data
(60) Provisional application No. 62/393,680, filed on Sep. 13, 2016.

(51) Int. Cl. A61M 5/00; A61B 5/026; A61B 6/00; A61B 8/08; A61B 5/00; A61B 8/00

(52) U.S. Cl. CPC A61M 5/007; A61B 5/0263; A61B 6/481; A61B 8/481; A61B 6/541; A61M 2230/04; A61B 8/543; A61B 6/469; A61B 8/469; A61B 5/7267; A61M 2205/50; A61B 5/748

(57) ABSTRACT

Systems and methods are provided for imaging a patient. Target imaging parameters for imaging a region of interest of a patient are determined based on desired attributes of images to be generated for the region of interest. Administration parameters for administering a contrast agent are determined based on the target imaging parameters using a computational model of blood flow and contrast agent circulation. A trigger time for imaging the region of interest is determined based on the administration parameters using the computational model of blood flow and contrast agent circulation. The region of interest of the patient is caused to be imaged based on the administration parameters and the trigger time.

### CROSS-REFERENCE TO RELATED APPLICATIONS

This application claims the benefit of U.S. Provisional Application No. 62/393,680, filed Sep. 13, 2016, the disclosure of which is herein incorporated by reference in its entirety.

### BACKGROUND OF THE INVENTION

The present invention relates generally to medical imaging using contrast agents, and more particularly to optimizing the contrast imaging of a patient to minimize patient exposure to radiation and maximize image quality.

Medical imaging systems are commonly used by doctors and other medical professionals for clinical analysis and medical intervention procedures. The contrast of structures or fluids of the patient can be enhanced in the medical imaging by injecting a contrast agent into the patient. In order to minimize the patient's exposure to radiation, imaging should not be started until the contrast agent reaches the target region of interest of the patient. However, conventional approaches for determining the time that the contrast agent will reach the target region of interest of the patient typically result in unnecessary patient exposure to radiation or are inaccurate.

One conventional approach to determine the time that the contrast agent will reach the target region of interest involves injecting the patient with a contrast agent while imaging the patient to observe when the contrast agent bolus reaches the target region of interest. Another conventional approach to determine the time that the contrast agent will reach the target region of interest involves estimating the time based on the patient's characteristics (e.g., height, weight, body surface area, etc.) and other physiological considerations (e.g., cardiac output, site of injection, renal function, etc.). Conventional approaches to determine the time that the contrast agent will reach the target region of interest result in unnecessary patient exposure to radiation and are typically inaccurate, thereby resulting in inconsistent image quality.

### BRIEF SUMMARY OF THE INVENTION

In accordance with one or more embodiments, systems and methods are provided for imaging a patient. Systems and methods are provided for imaging a patient. Target imaging parameters for imaging a region of interest of a patient are determined based on desired attributes of images to be generated for the region of interest. Administration parameters for administering a contrast agent are determined based on the target imaging parameters using a computational model of blood flow and contrast agent circulation. A trigger time for imaging the region of interest is determined based on the administration parameters using the computational model of blood flow and contrast agent circulation. The region of interest of the patient is caused to be imaged based on the administration parameters and the trigger time.

In accordance with one or more embodiments, the computational model for the patient is a computational fluid dynamics model personalized to model blood flow and contrast agent circulation of the patient. The computational fluid dynamics model may be personalized based on medical imaging data of the patient. The computational fluid dynamics model may also be personalized based on an anatomical model. The anatomical model may be generated by acquiring three-dimensional surface imaging data of the patient; generating a surface model of the patient from the three-dimensional surface imaging data; and matching the surface model of the patient to an anatomical model to provide the medical imaging data of the patient.

In accordance with one embodiment, the computational model for the patient is a machine learning based model trained to predict blood flow and contrast agent circulation of the patient.

In one embodiment, the machine learning based model may be trained by generating synthetic training data comprising patient characteristics and administration parameters; simulating blood flow and contrast media circulation for the synthetic training data using a computational fluid dynamics model; determining blood flow and contrast media circulation parameters for the synthetic training data from the simulating; extracting features from the synthetic training data; and training the machine learning based model to predict the blood flow and contrast media circulation parameters using the features extracted from the synthetic training data. The blood flow and contrast media circulation parameters may be predicted by generating a patient-specific anatomical model specific to the patient. In one embodiment, the patient-specific anatomical model is generated acquiring three-dimensional surface imaging data of the patient. A surface model of the patient is generated from the three-dimensional surface imaging data. The surface model of the patient is matched to an anatomical model to provide the medical imaging data of the patient. Features are extracted from the generated patient-specific anatomical model and the blood flow and contrast media circulation parameters are predicted using the features extracted from the patient-specific anatomical model. The administration parameters and the trigger time are determined based on the predicted blood flow and contrast media circulation parameters.

In another embodiment, the machine learning based model may be trained by generating synthetic training data comprising patient characteristics, administration parameters, and regions of interest. Blood flow and contrast media circulation for the synthetic training data is simulated using a computational fluid dynamics model. Times for imaging the regions of interest are determined from the simulations. Features are extracted from the synthetic training data. The machine learning based model is trained to predict the times for imaging the regions of interest using the extracted features.

In accordance with one or more embodiments, the administration parameters are determined as the administration parameters that result in a minimum error between the target imaging parameters and computed imaging parameters, where the computed imaging parameters are computed from the computational model of blood flow and contrast agent circulation using the administration parameters.

In accordance with one or more embodiments, the trigger time is determined by determining concentration levels of the contrast agent in the region of interest using the computational model of blood flow and contrast agent circulation and the administration parameters. The time that the concentration levels is at its maximum is determined to be the trigger time.

In accordance with one or more embodiments, causing the region of interest of the patient to be imaged based on the administration parameters and the trigger time includes at least one of automatically initiating imaging of the region of interest of the patient at the trigger time or notifying a user to manually initiate imaging of the region of interest of the patient at the trigger time.

These and other advantages of the invention will be apparent to those of ordinary skill in the art by reference to the following detailed description and the accompanying drawings.

### BRIEF DESCRIPTION OF THE DRAWINGS

FIG. 1 shows an exemplary system for optimizing contrast imaging of a patient, in accordance with one or more embodiments;

FIG. 2 shows a method for imaging a subject, in accordance with one or more embodiments;

FIG. 3 shows a graph of the relationship between enhancement of an image and concentration of a contrast agent for different tube voltage settings, in accordance with one or more embodiments;

FIG. 4 shows a workflow for training and applying a machine learning model for determining imaging parameters for imaging a region of interest, in accordance with one or more embodiments;

FIG. 5 shows a full-body systemic arterial model of a patient, in accordance with one or more embodiments;

FIG. 6 depicts a change in levels of concentration of a contrast agent over time, in accordance with one or more embodiments;

FIG. 7 shows an exemplary time-varying flow rate of a contrast agent over time, in accordance with one or more embodiments;

FIG. 8 shows a method for generating an anatomical model from a surface model of a patient, in accordance with one or more embodiments;

FIG. 9 shows a process for generating a surface model of a patient, in accordance with one or more embodiments;

FIG. 10 shows a workflow for training and applying a machine learning model for matching a surface model to an anatomical model, in accordance with one or more embodiments;

FIG. 11 shows a workflow for training and applying a machine learning model for predicting blood flow and contrast agent circulation parameters of a patient, in accordance with one or more embodiments;

FIG. 12 shows a workflow for training and applying a machine learning model for predicting a trigger time for initiating imaging of a patient, in accordance with one or more embodiments;

FIG. 13 shows a workflow for training and applying a machine learning model for determining imaging parameters and administration parameters, in accordance with one or more embodiments; and

FIG. 14 shows a high-level block diagram of a computer, in accordance with one or more embodiments.

### DETAILED DESCRIPTION

The present invention generally relates to optimizing contrast imaging of a patient by determining a trigger time to initiate imaging of a patient in order to minimize patient exposure to radiation. Embodiments of the present invention are described herein to give a visual understanding of methods for optimizing contrast imaging of a patient. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Further, it should be understood that while the embodiments discussed herein may be discussed with respect to medical imaging of a patient, the present invention is not so limited. Embodiments of the present invention may be applied for any type of imaging for any subject.

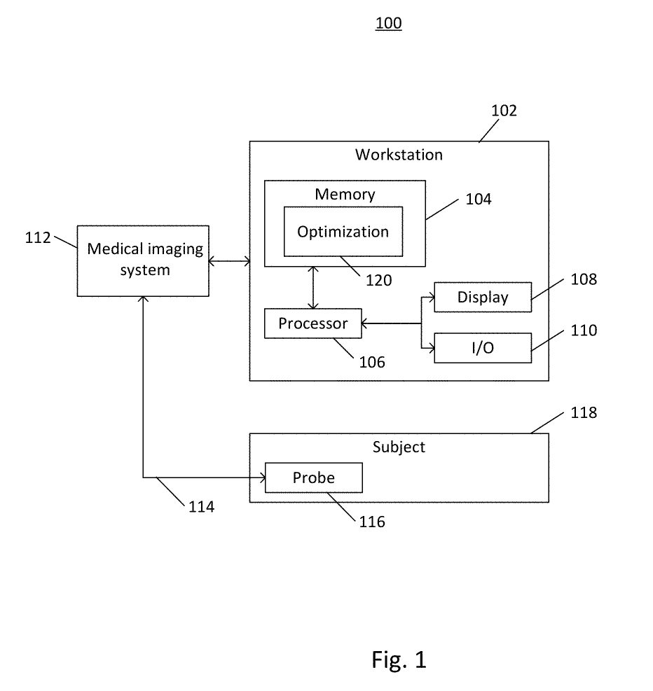

FIG. 1 shows a system 100 configured for optimizing contrast imaging of a patient, in accordance with one or more embodiments. System 100 includes workstation 102, which may be used for assisting a user (e.g., a doctor, clinician, or any other medical professional) during a procedure, such as, e.g., a patient examination. Workstation 102 includes one or more processors 106 communicatively coupled to memory 104, display device 108, and input/output devices 110. Memory 104 may store a plurality of modules representing functionality of workstation 102 performed when executed on processor 106. It should be understood that workstation 102 may also include additional elements, such as, e.g., a communications interface.

In one embodiment, an optimization module 120 is implemented on workstation 202 to optimize contrast imaging of a patient by determining a trigger time to initiate imaging of the patient. Optimization module 120 may be implemented as computer program instructions (e.g., code), which may be loaded into memory 104 and executed by processor 108.

Workstation 102 may assist the clinician in imaging subject 118 (e.g., a patient) for a medical procedure. Workstation 102 may receive medical imaging data generated by medical imaging system 112. Medical imaging system 112 may be of any modality, such as, e.g., x-ray, magnetic resonance imaging (MRI), computed tomography (CT), ultrasound (US), single-photon emission computed tomography (SPECT), positron emission tomography (PET), or any other suitable modality or combination of modalities.

In some embodiments, medical imaging system 112 may employ one or more probes 116 for imaging subject 118. Probe 116 may be instrumented with one or more devices (not shown) for performing the medical procedure. The devices instrumented on probe 116 may include, for example, imaging devices, tracking devices, insufflation devices, incision devices, and/or any other suitable device. Medical imaging system 112 is communicatively coupled to probe 116 via connection 114, which may include an electrical connection, an optical connection, a connection for insufflation (e.g., conduit), or any other suitable connection.

To enhance the visibility of blood vessels or organs of subject 118, a contrast agent (e.g., iodine, barium, gadolinium, microbubble, etc.) may be injected into subject 118 prior to medical imaging system 112 imaging subject 118. In order to optimize imaging and minimize exposure of subject 118 to radiation, medical imaging system 112 should not begin imaging subject 118 until the contrast agent reaches a target region of interest of subject 118 (e.g., the concentration of the contrast agent at the target region of interest is at its maximum). Advantageously, optimization module 120 applies a computational model to determine a trigger time to initiate imaging of subject 118 by medical imaging system 112 upon injecting the contrast agent into subject 118, thereby providing for improvements in computer related technology. The trigger time represents the interval of time between the start of the injection of the contrast agent and the initiation of the scan by medical imaging system 112. Optimizing module 120 may thus cause medical imaging system 112 to initiate imaging of subject 118, e.g., by automatically imaging subject 118 in accordance with the trigger time or by causing a user (e.g., via a notification) to image subject 118 in accordance with the trigger time.

Optimization module 120 determines the trigger time for initiating imaging of subject 118 to reduce patient exposure to radiation and avoid the inaccuracies associated with conventional systems.

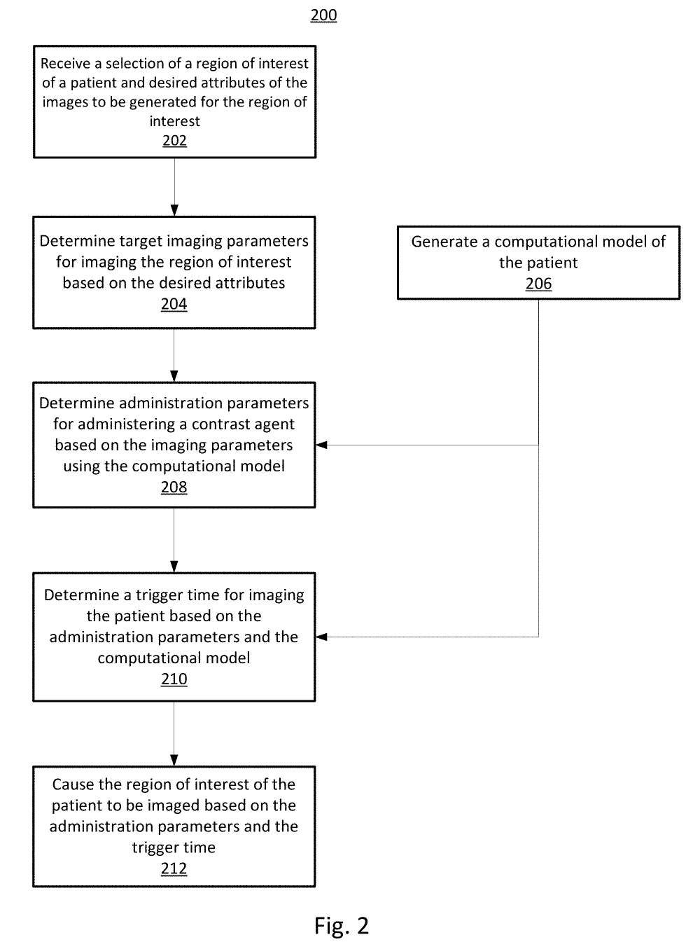

FIG. 2 shows a method 200 for imaging a subject, in accordance with one or more embodiments. Method 200 will be discussed with respect to system 100 of FIG. 1. In one embodiment, optimization module 120 implemented on workstation 102 of FIG. 1 performs at least some of the steps of method 200 of FIG. 2.

At step 202, a selection of a region of interest of a patient (e.g., subject 118), and desired attributes of the images to be generated for the region on interest, are received from a user (e.g., a doctor). The selected region of interest and desired attributes may be defined by the user using display 108 and/or input/output device 110 of FIG. 1. Other examination characteristics may also be received at step 202.

In one embodiment, the region of interest of the patient may be an organ or other structure of the patient. For example, the region of interest may be the heart or an artery of the patient. In another embodiment, the region of interest is automatically determined based on a diagnostic/clinical issue input (by the user) for which the imaging is being performed. For example, the diagnostic/clinical issue may be ruling out coronary artery disease, ruling out a pulmonary embolism, sizing a stent graft, etc.

As noted, desired attributes of the images to be generated for the region on interest are also received. The desired attributes may be defined according to the user's preferences of the characteristics of the generated images. Examples of the desired attributes may include image enhancement (e.g., measured in Hounsfield units), radiation dose, image noise, slice thickness, high or low contrast resolution, etc. The slice thickness is typically linked to table speed, such that higher table speeds results in larger slice thickness. Further, there is generally a tradeoff between image quality and radiation dose, which is application (i.e., scan) dependent. In one embodiment, the desired attributes are specified by applying an algorithm based on the region of interest.

At step 204, target imaging parameters for imaging the region of interest are determined based on the desired attributes of the images to be generated for the region on interest. The imaging parameters for imaging the region of interest may include, for example, parameters of the contrast agent (e.g., the concentration of the contrast agent in the region of interest) and parameters of the medical imaging system (e.g., tube voltage, tube current, exposure time (i.e., duration of scan), table speed, the reconstruction algorithm (e.g., filter, convolution kernel properties, etc.), medical imaging system specifications (e.g., beam spectra, geometry, x-ray beam collimation, etc.), etc.). The characteristics of the patient may include, e.g., weight of the patient, height of the patient, surface area of the patient, etc. The imaging parameters for imaging the region of interest may be determined using any suitable approach.

In one embodiment, a transfer function may be applied to determine the imaging parameters for imaging the region of interest as an output given the desired attributes as an input. A transfer function is a mathematical representation of the relationship between the input desired imaging attributes and the output parameters for imaging the region of interest. The mathematical relationship may be defined as mathematical formulas derived from, e.g., physics based models or from experiments. In one embodiment, the transfer function may be determined as is known in the art. The transfer function may be represented as follows in Equation (1).

$$( \begin{matrix}concentration \\ tube voltage \\ tube current \\ exposure time \\ table speed\end{matrix} ) = f ( \begin{matrix}enhancement \\ radiation dose \\ image noise \\ slice thickness \\ contrast \\ reconstruction algorithm \\ scanner specifications\end{matrix} ) . \qquad ( 1 )$$

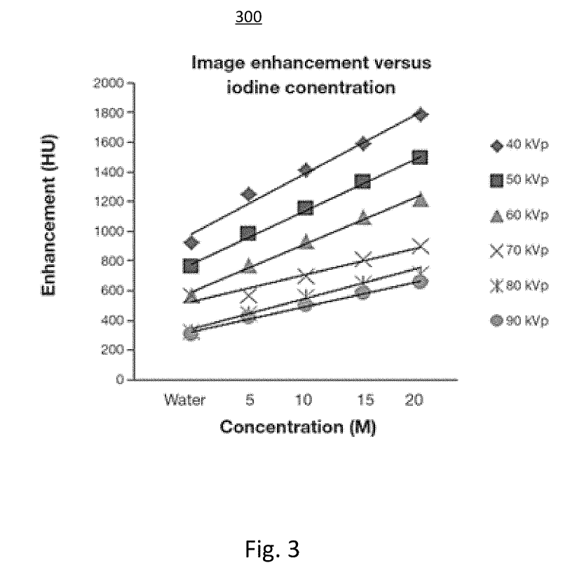

In another embodiment, the imaging parameters for imaging the region of interest may be determined based on mappings between the input desired attributes and the output imaging parameters for imaging the region of interest. The mappings may be generated based on, e.g., studies identifying the relationship between the desired attributes and the imaging parameters for imaging the region of interest and stored in a database (not shown in FIG. 1). In one embodiment, the mappings may additionally or alternatively be generated based on patient specific data, synthetic data generated by simulating a medical imaging system, and/or data generated from in vitro experiments. FIG. 3 illustratively shows a graph 300 of the relationship between the enhancement of the image (in Hounsfield units) and the concentration of the contrast agent (e.g., iodine) in the region of interest for different tube voltage settings.

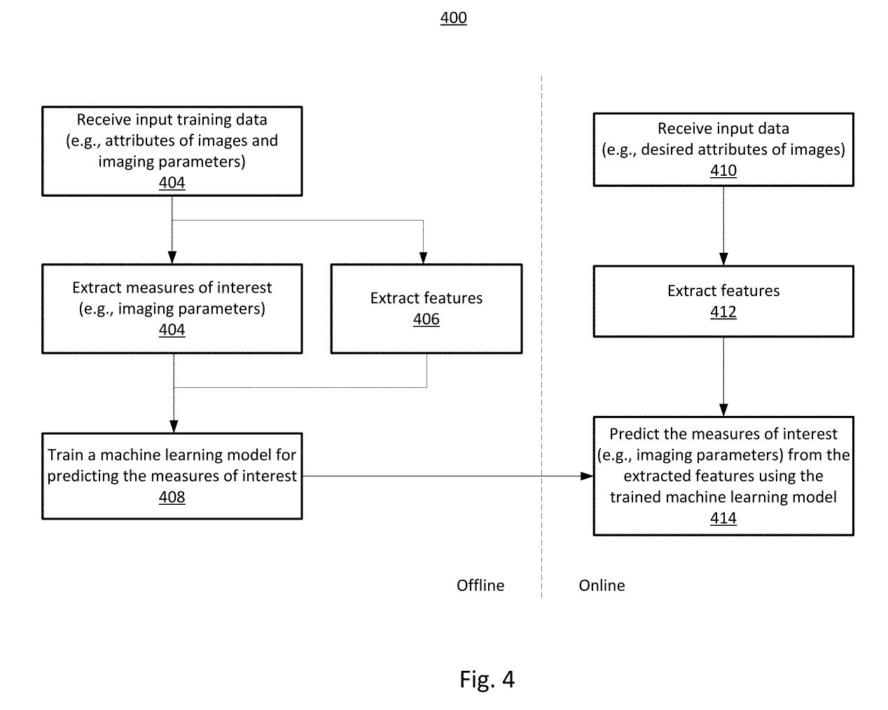

A number of studies may be performed to populate the database, which can then be used to derive the values of the imaging parameters for imaging the region of interest based on the desired attributes of the images to be generated for the region of interest. In one embodiment, the values of the imaging parameters for imaging the region of interest may be determined as the values of the imaging parameters that result in attributes of the images closest to the desired attributes. In another embodiment, the values of the imaging parameters for imaging the region of interest may be determined using one or more machine learning algorithms trained to learn the mappings between the imaging parameters for imaging the region of interest and the desired attributes of the images to be generated for the region of interest. In one example, the trained machine learning algorithm may be trained and applied in accordance with FIG. 4.

FIG. 4 shows a workflow 400 for training and applying a machine learning model for determining imaging parameters for imaging a region of interest, in accordance with one or more embodiments. In one embodiment, the trained machine learning model resulting from workflow 400 may be applied to determine the imaging parameters in step 204 of FIG. 2. Blocks 402-408 show an offline or training stage for training a machine learning model and blocks 410-414 show an online stage for applying the trained machine learning model.

During an offline training stage, at step 402, input training data is received. The input training data may be any suitable data for training a machine learning model to predict measures of interest. For example, the input training data may include attributes of training images, such as, e.g., parameters of the contrast agent (e.g., the concentration of the contrast agent in the region of interest) and parameters of the medical imaging system (e.g., tube voltage, tube current, exposure time (i.e., duration of scan), table speed, the reconstruction algorithm (e.g., filter, convolution kernel properties, etc.), medical imaging system specifications (e.g., beam spectra, geometry, x-ray beam collimation, etc.), etc. The input training data may also include known imaging parameters for imaging the region of interest in the training images. The input training data may be received from a database populated with actual data of one or more patients, synthetic data generated by simulating a medical imaging system, and/or data generated from in vitro experiments.

At step 404, measures of interest are extracted from the input data. In this embodiment, the measures of interest include imaging parameters for imaging the region of interest. At step 406, features are extracted from the input training data. Exemplary features may include enhancement, radiation dose, image noise, slice thickness, high/low contrast, reconstruction algorithm, scanner specifications, and any linear or non-linear combinations of the above. The features are determined based on the characteristics available (e.g., in the database) for the input training data. It should be understood that step 406 may be performed at any time prior to step 408 (e.g., before step 404, after step 404, or concurrently with (e.g., in parallel with) step 404). At step 408, one or more machine learning models are trained to predict the measures of interest (i.e., imaging parameters for imaging the region of interest). The machine learning approaches may include, e.g., regression, instance-based methods, regularization methods, decision tree learning, Bayesian, kernel methods, clustering methods, association rule learning, artificial neural networks, dimensionality reduction, ensemble methods, or any other suitable machine learning approach. In one embodiment, the machine learning models may be trained using methods known in the art.

During the online stage, at step 410, input data is received. The input data received at this step represents unseen data of the patient to be imaged. The input data may be any suitable data for predicting the measures of interest by a trained machine learning model. For example, input data at step 410 may include the desired attributes of images to be generated (e.g., enhancement, radiation dose, image noise, slice thickness, contrast resolution, etc.). In one embodiment, the desired attributes of the images received at step 410 of FIG. 4 is the desired attributes of the images received at step 202 of FIG. 2. At block 412, features are extracted from the input data. At block 414, measures of interest are predicted from the (i.e., based on the) extracted features using the trained machine learning model. The measures of interest may include the imaging parameters for imaging the region of interest. In one embodiment, the imaging parameters determined from the trained machine learning model resulting from workflow 400 may be the imaging parameters determined at step 204 of FIG. 2.

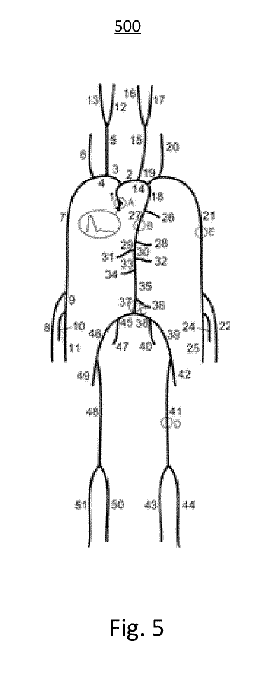

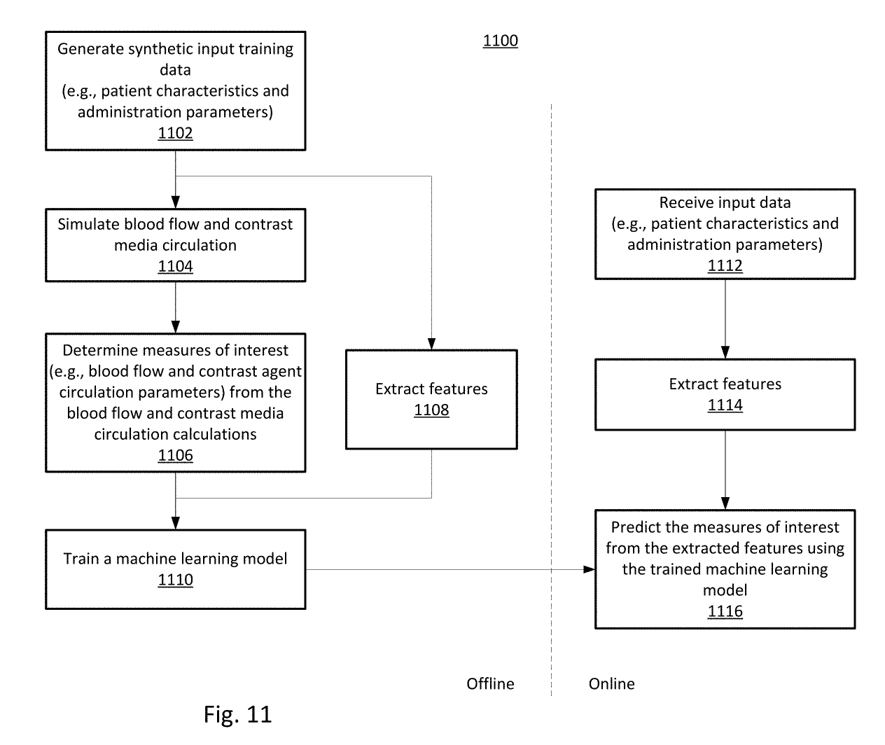

Referring back to FIG. 2, at step 206, a computational model of the patient is generated. The computational model may include any suitable computational model that models the blood flow and contrast agent circulation of a patient to provide blood flow and contrast agent circulation parameters, such as, e.g., flow, velocities, contrast agent concentration, etc. In one embodiment, the computational model may be a computational fluid dynamics model personalized for a specific patient, as discussed below with respect to FIG. 5. In another embodiment, the computational model may be a machine learning model that predicts the blood flow and contrast agent circulation of a patient, as discussed below with respect to FIG. 11. It should be understood that step 206 may be performed at any time prior to step 208 (e.g., before steps 202-204, after steps 202-204, or concurrently with (e.g., in parallel with) steps 202-204).

At step 208, administration parameters for administering a contrast agent are determined based on the imaging parameters for imaging the region of interest (as determined at step 204) using the generated computational model. The administration parameters for administering the contrast agent may include, e.g., the volume of the contrast agent to be administered or injected into the patient, the concentration of the contrast agent to be injected into the patient, and the rate and profile of the injection of the contrast agent into the patient. The administration parameters for administering the contrast agent may be determined using any suitable approach.

In one embodiment, a parameter estimation problem may be formulated as a solution to a system of nonlinear equations, with each equation representing the residual error between the computed and desired (i.e., reference) values of the imaging parameters for imaging the region of interest (e.g., the concentration of the contrast agent in the region of interest, the scan exposure time, etc.). The system of nonlinear equations may be represented as the following system of nonlinear equations f(xᵢ) in Equation (2) to estimate administration parameters xᵢ representing the characteristics for administering the contrast agent (e.g., injected volume, concentration, and injection rate/profile), where each equation represents the residual error between the computed and desired values of the administration parameters for imaging the region of interest (e.g., the concentration of the contrast agent in the region of interest, and the scan exposure time).

$$f ( \begin{matrix}injected volume \\ concetration \\ injection rate / profile\end{matrix} ) = \{ \begin{matrix}{( Rol concentration )}_{comp} - {( Rol concentration )}_{ref} \\ {( scan time )}_{comp} - {( scan time )}_{ref}\end{matrix} \} = \{ \begin{matrix}0 \\ 0\end{matrix} \} \qquad ( 2 )$$

The administration parameters are obtained by running the parameter estimation problem (i.e., running the computational model) iteratively with different administration parameter values until the computed values match the desired reference values. For example, when the blood flow and contrast agent propagation are computed for a given set of administration parameter values, the concentration (one of the measure computed by the computational model) in the ROI is determined as a function of time, and can be compared with the reference value determined at the previous step.

The calibration method could automatically estimate administration parameters xᵢ (i.e., the characteristics for administering the contrast agent) to ensure that the computed values of the imaging parameters for imaging the region of interest minimize the objective function. Any suitable bolus shaping approach may be employed for administering the contrast agent. For example, exponential bolus shaping may be applied to result in a more uniform contrast enhancement.

At step 210, the trigger time for imaging the patient is determined based on the administration parameters for administering the contrast agent using the computational model. The trigger time represents the interval of time between the start of the injection and the initiation of the scan. For example, the trigger time can be 20 seconds. To determine the trigger time, the computational model (generated at step 206) is run one or more times to determine the time (e.g., relative to the start of the injection of the contrast agent) at which the concentration of the contrast agent in the region of interest is the highest, based on the administration parameters for administering the contrast agent. For example, the parameter estimation problem (i.e., the computational model) may be iteratively solved with different trigger times until the time that the concentration of the contrast agent in the region of interest is at its highest. In one embodiment, the time delay for image acquisition is also taken into account (e.g., the time required for the table to be put in place, to instruct the patient to hold his or her breath, etc.).

At step 212, the region of interest of the patient is caused to be imaged based on the administration characteristics for administering the contrast agent and the trigger time. For example, in one embodiment, imaging of the region of interest of the patient is automatically initiated (e.g., by optimization module 120 of FIG. 1) after the trigger time has elapsed upon administering the contrast agent to the patient in accordance with the administration parameters. In another embodiment, imaging of the region of interest of the patient is caused to be manually initiated by a user, e.g., via a notification or any other indication or instruction (e.g., from optimization module 120 of FIG. 1).

Computational Fluid Dynamics Computational Model

FIG. 5 shows a full-body systemic arterial model 500 (i.e., an arterial geometry) of a patient, in accordance with one or more embodiments. As shown in FIG. 4, the full-body systemic arterial model 400 includes 51 arteries (numbered 1-51)

A population averaged whole-body systemic arterial model may be used as a starting point. For example, the population averaged whole body systemic arterial model may be based on an atlas model or previously published arterial models. Computational modeling algorithms (e.g., computational fluid dynamics (CFD) algorithms) may be applied to simulate blood flow and contrast agent circulation (or any other patient-specific hemodynamics). The lengths and cross-section of the arteries in the systemic arterial model are then personalized based on the initialization measurement data (e.g., medical imaging data and/or initial non-imaging measurements) of the patient for patient-specific simulation of blood flow and contrast agent circulation in the patient. In one embodiment, the arterial model 500 is the computational model generated at step 206 of FIG. 2.

Arterial model 500 may be derived from the Navier-Stokes equations using appropriate assumptions on the nature of the flow in blood vessels. In healthy, non-stenotic coronary arteries, the flow is assumed to have a dominant component in the axial direction, and axial symmetry. For axisymmetric flow, the continuity equation is:

$$\frac{\partial {u}_{x}}{\partial x} + \frac{1}{r} \frac{\partial ( {ru}_{r} )}{\partial r} = 0 \qquad ( 3 )$$

where x is the coordinate in the longitudinal direction, u is the velocity, and r is the radius. By integrating over the cross-sectional area, while accounting for the changing vessel cross-sectional area, the following formulation is obtained:

$$\frac{\partial A ( x , t )}{\partial t} + \frac{\partial q ( x , t )}{\partial x} = 0 \qquad ( 4 )$$

where A(x,t) is the cross-sectional area and q(x,t) is the flow rate.

For the momentum equations, it is assumed that the pressure is constant within each cross-section, varying primarily along the longitudinal direction. The axial momentum equation is as follows:

$$\frac{\partial {u}_{x}}{\partial t} + u \frac{\partial {u}_{x}}{\partial x} + {u}_{r} \frac{\partial {u}_{x}}{\partial r} + \frac{1}{ρ} \frac{\partial p}{\partial x} = \frac{υ}{r} \frac{\partial }{\partial r} ( r \frac{\partial {u}_{x}}{\partial r} ) \qquad ( 5 )$$

where p is the pressure, ρ is the density, and u is the kinematic viscosity. By integrating over the cross-sectional area, the following formulation is obtained:

$$\frac{\partial q ( x , t )}{\partial t} + \frac{\partial }{\partial x} ( α \frac{{q}^{2} ( x , t )}{A ( x , t )} ) + \frac{A ( x , t )}{ρ} \frac{\partial p ( x , t )}{\partial x} = {K}_{R} \frac{q ( x , t )}{A ( x , t )} \qquad ( 6 )$$

where the coefficients α and K_{R} account for the momentum-flux correction and viscous losses due to friction, respectively.

To close the system of equations, a state equation is needed to relate the pressure inside the vessel to the cross-sectional area. The vessel wall is modeled as a purely elastic material as in Equation (7), which responds to changing pressures through radial displacements:

$$p ( x , t ) = {Ψ}_{el} ( A ) + {p}_{0} = \frac{4}{3} \frac{Eh}{{r}_{0}} ( x ) ( 1 - \sqrt{\frac{{A}_{0} ( x )}{A ( x , t )}} ) + {p}_{0} \qquad ( 7 )$$

where E is the Young modulus, h is the wall thickness, rₒ is the initial radius corresponding to the initial pressure pₒ, and Aₒ is the initial cross-sectional area.

The elastic wall properties may be estimated using best fit experimental data. The system of equations may be discretized with a finite difference, finite element, or finite volume method. At each bifurcation, the continuity of flow qₚ and total pressure pₚ is imposed in Equations (8) and (9), respectively.

$$\begin{matrix}{q}_{p} = {\sum }_{i} {( {q}_{d} )}_{i} & ( 8 ) \\ {p}_{p} + \frac{1}{2} ρ \frac{{q}_{p}^{2}}{{A}_{p}^{2}} = {( {p}_{d} )}_{i} + \frac{1}{2} ρ \frac{{( {q}_{d}^{2} )}_{i}}{{( {A}_{d}^{2} )}_{i}} & ( 9 )\end{matrix}$$

where subscript p refers to the parent and subscript d refers to the daughter vessels.

To close the system of equations at inlets and outlets, proper boundary conditions are provided. The outlets are typically lumped parameter models, leading to the multiscale formulation of the CFD model.

In accordance with one embodiment, the CFD model is personalized based on patient characteristics for that specific patient to allow for patient-specific computations of blood flow and contrast agent circulation parameters, such as, e.g., flow, velocities, contrast agent concentration, etc. For example, patient-specific boundary conditions for the arterial geometry, arterial wall properties, inlet boundary condition, and outlet boundary conditions (i.e., parameters of the models coupled at the outlet of each terminal artery) may be defined to personalize the CFD model.

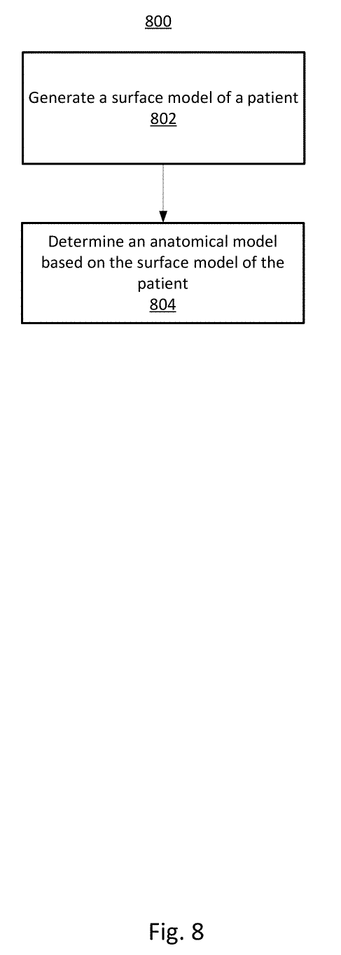

Arterial Geometry: The arterial geometry may be personalized based on imaging data of the patient (of the entire body or of a portion of the body). For example, the imaging data of the patient may be medical imaging data of the patient. The medical imaging data may be any medical images of the patient depicting the interior of the patient, such as, e.g., x-ray, MRI, ultrasound, etc. A three-dimensional (3D) anatomical model of the patient may be generated from the medical imaging data (e.g., by segmentation) and arterial geometries of the patient are extracted from the 3D anatomical model. In another example, medical imaging data from a topogram (i.e., a scout scan) is generated and compared with a database of stored medical imaging data (e.g., high-resolution computed tomography imaging data) from which arterial geometries of the patient are extracted. In yet another example, the imaging data of the patient may be surface imaging data of the patient. A surface model of the patient may be estimated using a 3D camera and compared with the database of stored imaging data (e.g., CT imaging data) to arterial geometries of the patient. Alternatively, an algorithm may match the surface model of the patient to a 3D vessel model from a database of 3D vessel models to determine the arterial geometries of the patient. The determination of arterial geometries based on a surface model of the patient is further described below with respect to FIG. 8. In one embodiment, a 3D model of a patient is generated from previously generated medical imaging data if previously generated medical imaging data exists; however if no previously generated medical imaging data exists, a surface imaging data of the patient is acquired to determine the arterial geometries of the patient. In this manner, patient exposure to radiation is reduced.

In another embodiment, arterial geometries may be estimated from physiological qualities of the patient, such as, e.g., weight, height, body surface area, age, sex, cardiac output, etc. The physiological qualities of the patient are compared to a plurality of medical imaging data (e.g., CT imaging data or 3D vessel models) associated with physiological qualities data and stored in a database. Arterial geometries of the patient are determined based on the medical imaging data that is associated with physiological qualities data that most closely matches the physiological qualities of the patient. In some embodiments, the physiological qualities of the patient are input directly to the computational model to determine the arterial geometries of the patient.

Arterial Wall Properties: The arterial wall properties may be derived from, e.g., brachial-ankle oximetry or pressure measurements (performed at the brachial artery and in the posterior tibial and the dorsalis pedis arteries at each ankle). Measurements may alternatively or additionally be performed at other arterial locations (e.g., femoral artery or the carotid artery). Based on the transit time estimated from these measurements, several localized pulse wave velocities may be determined and then used to define the arterial wall properties. The more measurements that are available, the more reliable the personalization will be. In one embodiment, an electrocardiogram (ECG) signal may additionally be used for providing a reference signal for the transit time computations.

Inlet Boundary Conditions: Depending on the availability of in-vivo measurements and the underlying assumptions used in the models, the one or more of the following inlet boundary conditions may be used: a time-varying flow profile, a lumped model of the heart coupled at the inlet, or a non-reflecting boundary condition like a forward running pressure wave. A time-varying velocity profile (or flow rate profile) can be consistently determined in a clinical setting, and is often part of the diagnostic workflow (e.g., a 2D/3D phase-contrast MRI or Doppler ultrasound). The parameters of the lumped model of the heart can be computed based on non-invasively acquired flow rate and pressure values. Alternatively, the cardiac output may be derived from non-invasive signals and used together with the heart rate to scale a population-average aortic inlet profile, so as to provide a personalized flow profile at the inlet of the ascending aorta. Furthermore, the cardiac output (or even the flow velocity in the ascending aorta) may be derived from previously performed medical imaging data (e.g., echocardiogram or MRI).

Outlet Boundary Conditions: Outlet boundary conditions may be classified as either periodic or non-periodic boundary conditions. Periodic boundary conditions can only be used in steady-state computations (e.g., the patient state does not change from one heart cycle to the next—the same inlet flow rate profile is applied for each heart cycle) and require flow information from the previous heart cycle. Non-periodic boundary conditions do not have such restrictions (e.g., they can be used to model the transition from a rest state to an exercise-hyperemic state for a patient).

Two physiologically motivated boundary conditions may be used for patient-specific computations.

The first is a three-element Windkessel model (WK) may be used:

$$\frac{\partial p}{\partial t} = {R}_{p} \frac{\partial q}{\partial t} - \frac{p}{{R}_{d} \cdot C} + \frac{q ( {R}_{p} + {R}_{d} )}{{R}_{d} \cdot C} \qquad ( 10 )$$

where Rₚ is the proximal resistance, R_{d} is the distal resistance, C is the compliance, and p and q refer to the pressure and flow rate at the inlet of the Windkessel model, respectively.

The second is a structured tree model (ST). The structured tree is a binary, asymmetrical vascular tree computed individually for each outlet, composed of a varying number of vessel generations. It is terminated once the radius decreases below a preset minimum radius and its root impedance, z(t), is computed recursively. The root impedance is applied at the outlet of the proximal domain through a convolution integral:

$$p ( x , t ) = {\int }_{t - T}^{t} q ( x , τ ) z ( x , t - τ ) d τ \qquad ( 11 )$$

where T is the period.

For the outlet boundary conditions, the total compliance may be determined from the pulse pressure information derived from the brachial-ankle measurements. The total compliance can then be distributed to the outlets based on the size of the terminal arterial segments. The resistance at each outlet can be determined from apriori defined flow distributions, from body part/organ sizes, etc.

The personalization framework may involve one or more steps of direct parameter computation or iterative parameter computation. In one embodiment, the personalization framework comprises two sequential steps. First, a series of parameters are computed directly, and next, a fully automatic optimization-based calibration method is employed to estimate the values of the remaining parameters, ensuring that the personalized computations match the measurements. The parameter estimation problem is formulated as a numerical optimization problem, the goal of which is to find a set of parameter values for which the objectives are met. The measurements used at the second step may be the non-invasive blood pressure measurements performed at the various arterial locations.

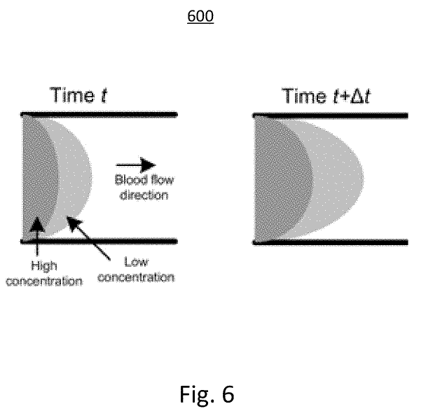

The flow computation step includes a model of contrast agent propagation. The transport of the contrast agent is determined by diffusion and advection. For laminar axisymmetric flow, the time-varying concentration at any location and time is given by:

$$\frac{\partial C ( r , l , t )}{\partial t} = D \cdot ( \frac{{\partial }^{2} C ( r , l , t )}{\partial {r}^{2}} + \frac{1}{r} \cdot \frac{\partial C ( r , l , t )}{\partial r} + \frac{{\partial }^{2} C ( r , l , t )}{\partial {l}^{2}} ) - v ( r ) \frac{\partial C ( r , l , t )}{\partial l} \qquad ( 12 )$$

where C is the concentration, r is the radial length, l is the longitudinal length, t is the time, and v is the velocity. The velocity v(r) is provided by the blood flow computational model. FIG. 6 illustratively shows the change in levels of concentration of the contrast agent over time in accordance with Equation (12), in one embodiment. Equation (12) can be further adapted if the radius is changing in time. Furthermore, since the flow distribution at bifurcations is determined by the blood flow model, the propagation of the contrast agent can be directly computed in branching arterial trees, without requiring any other assumption.

Two additional aspects are to be taken into account in the personalization framework: the injection protocol and the mixing of the flow rates of the blood and the contrast agent.

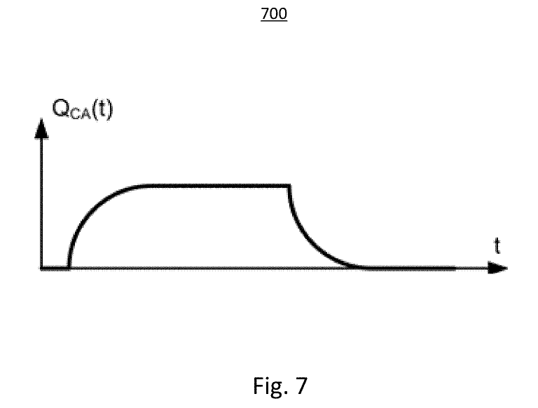

A rectangular function may be used to model the injection. In this case, the output of the catheter can be modeled through an analogy with electrical networks: a resistance and a compliance. Hence, the injection curve may be represented as follows:

$${Q}_{CA} ( t ) = \{ \begin{matrix}0 , t < {T}_{S} \\ Q \cdot ( 1 - {e}^{- ( t - {T}_{S} ) / {T}_{L}} ) , {T}_{S} \leq t \leq {T}_{S} + {T}_{D} \\ Q \cdot ( 1 - {e}^{- ( t - {T}_{S} ) / {T}_{L}} ) \cdot {e}^{- ( t - ( {T}_{S} + {T}_{D} ) ) / {T}_{L}} , t > {T}_{S} + {T}_{D}\end{matrix} \qquad ( 13 )$$

where Q_{CA}(t) is the time-varying flow rate of the contrast agent, T_{S} is the injection start time, T_{D} is the duration of the injection, and T_{L} is the time delay. FIG. 7 shows an exemplary time-varying flow rate Q_{CA}(t) of a contrast agent over time t, in accordance with one embodiment. It should be understood that any injection function may be used, particularly if an automatic injection tool is available. Manual injection profiles may be modeled by approximation.

Since the flow rate of the contrast agent augments the blood flow rate, the mixing of the two flow rates are taken into account in the blood flow computation:

Q_{T}(t)=Q_{B}(t)+Q_{CA}(t) (14)

where Q_{B}(t) is the blood flow rate and Q_{T}(t) is the total flow rate. The initial concentration at the location of injection is computed as the ratio between Q_{CA}(t) and Q_{B}(t).

Both the flow computation step and the contrast propagation step may be performed with other methods, which may be based on zero-, one-, two-, or three-dimensional spatial modeling.

As described above, a surface model of a patient may be used to estimate an anatomical model of the patient (e.g., where previously generated medical imaging data is not available). FIG. 8 shows an exemplary method 800 for generating an anatomical model of a patient from a surface model of the patient, in accordance with one or more embodiments. In one embodiment, method 800 may be used to generate an anatomical model for personalizing the arterial geometry is model 500 in FIG. 5.

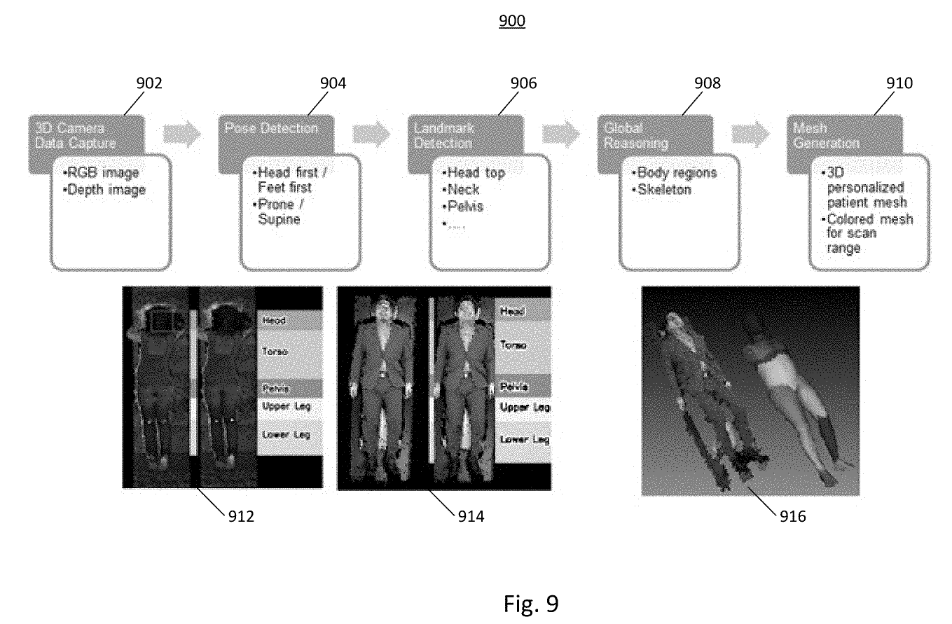

At step 802, a surface model of a patient is generated. In one embodiment, the surface model of the patient may be generated in accordance with the process 900 shown in FIG. 9 for generating a surface model of a patient. Generation of a surface model of a patient from surface imaging data is further described in U.S. Patent Publication No. 2017/0100089, the disclosure of which is incorporated herein by reference in its entirety.

At step 902 of FIG. 9, surface imaging data of a patient is acquired using one or more 3D imaging cameras. The 3D camera can be a structured light based camera (such as Microsoft Kinect or ASUS Xtion), a stereo camera, or a time of flight camera (such as Creative TOF camera). The acquired imaging data may include an RGBD image data (Red, Green, Blue+Depth). RGBD image data is an RGB image, in which each pixel has an RGB value, and a depth image, in which the value of each pixel corresponds to a depth or distance of the pixel from the camera. The color data in the RGB image and the depth (range) data in the depth image can be combined to represent the RGBD image data of the patient as a 3D point cloud in a reprojected image.

A depth camera for acquiring the depth image may be mounted on a gantry of a medical imaging system, the ceiling of the examination room, or any other suitable location. During the image acquisition, the patient may be lying on the scanner table or standing in front of the camera.

At step 904, pose detection is performed on the reprojected image to classify a pose of the patient. Given the coarse patient position information, the patient pose can be classified as head first versus feat first and classified as prone versus supine using one or more machine-learning based pose classifiers. Each of the pose classifiers can be a trained Probabilistic Boosting Tree (PBT) classifier.

At step 906, landmark detection is performed. Given the patient pose information, a sparse body surface model including a plurality of anatomical landmarks is fit to the reprojected image data. The body surface model can be represented as a Directed Acyclic Graph (DAG) over the anatomical landmarks on the body surface, where the graph captures the relative position of the landmarks with respect to each other. In an advantageous embodiment, the patient surface is modeled using 10 body landmarks—head, groin, and left and right landmarks for shoulders, waist, knees, and ankles. Respective landmark detectors are trained for each of the landmarks. For example, for each landmark, a multi-channel PBT classifier with Haar features extracted from the same channels as used to train the pose classifiers (e.g., reprojected depth image, surface normal data, saturation image, and U and V channels from Luv space) can be used to train each landmark detector.

At step 908, after all the landmark hypotheses for all the landmarks are obtained, a global reasoning is performed on the landmark hypotheses to obtain a set of landmarks with the highest joint likelihood based on the trained landmark detectors as well as the contextual information in the DAG. This sequential process of landmark detection handles the size and scale variations across patients of different ages. Once the final set of landmarks is detected using the global reasoning, body regions of the patient in the reprojected image can be defined based on the set of landmarks. For example, the reprojected image can be divided into body regions of head, torso, pelvis, upper leg, and lower leg. In a possible implementation, a human skeleton model can be fit the reprojected depth image based on the detected landmarks.

At step 910, a surface mesh of the patient is generated. The surface mesh may be a 3D personalized mesh of the surface of the patient. In some embodiments, the mesh may be colored to represent different scan ranges. The surface mesh may be constructed using any suitable method, such as, e.g., known reconstruction algorithms. The surface mesh may be represented as a 3D point-cloud, a 3D surface, a 3D level set, or as any other format representing a 3D geometric figure. Image 916 illustrative shows the generated surface mesh of the patient.

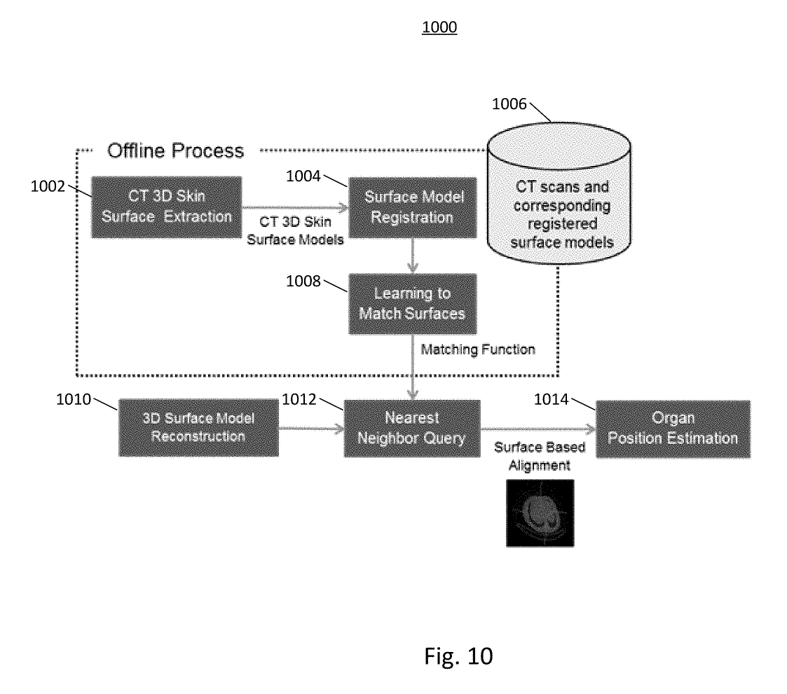

Returning back to FIG. 8, at step 804, an anatomical model is determined based on the surface model of the patient. The anatomical model may be used to estimate the position of organs in the patient. In one example, the surface model of the patient is compared with a database of medical imaging data (e.g., CT imaging data with corresponding topogram or a 3D anatomical model). The medical imaging data that most closely fits the surface model is identified to determine the anatomical model. In one embodiment, the surface model is matched against a database of medical imaging data to obtain a ranked list of anatomical models ordered based on their similarity to the surface model. In one embodiment, multiple databases can be used depending on the region of the body to be imaged. The surface model may be fit to an anatomical model using any suitable method. In one embodiment, machine learning algorithms (e.g., metric learning or deep learning) are used to learn a matching function to match a surface model to an anatomical model, such as, e.g., the machine learning model trained in FIG. 10.

FIG. 10 shows a workflow 1000 for training and applying a machine learning matching function for matching a surface model to an anatomical model, in accordance with one or more embodiments. Blocks 1002-1008 show an offline or training stage for training a machine learning matching function and blocks 1010-1014 show an online stage for applying the trained machine learning matching function to estimate organ position.

During the offline stage, at block 1002, a 3D surface model is extracted from medical imaging data (e.g., CT imaging data or anatomical models). The medical imaging data may be data of one or more patients or may be synthetic data generated by simulating a medical imaging system. The surface model may be extracted from the medical imaging data using any suitable approach, such as, e.g., methods known in the art. At block 1004, the extracted surface model is registered with database 1006 and stored with its associated medical imaging data in database 1006. At block 1008, one or more machine learning models are trained to match surface models to medical imaging data.

During the online stage, at block 1010, a surface model is received. The surface model may be the surface model received at step 802 of FIG. 8. At block 1012, medical imaging data (e.g., an anatomical model) that most closely matches (e.g., the nearest neighbor) the received surface model is determined using the trained matching function. At block 1014, organ position is estimated using the matching medical imaging data. The matching anatomical model may be used to personalize arterial geometry to provide a personalized CFD computational model, as described above with respect to FIG. 5.

Machine Learning-Based Computational Model

As described above with respect to FIG. 5, the computational model generated at step 206 in FIG. 2 may be a CFD computational model in one embodiment. However, since the CFD model is based on partial differential equations that can only be solved numerically, a large number of algebraic equations need to be solved, making it a computationally expensive approach. Solutions to these models may require hours of processing on powerful processing clusters to generate high-fidelity models representing complete 3D velocity fields.

In accordance with one embodiment, the computational model generated at step 206 of FIG. 2 may be a machine learning based model trained to predict blood flow and contrast agent circulation parameters in a patient. A relationship between input data is represented by a model built from a database of training samples with known characteristics and outcomes in an offline or training stage. Once the model is trained, in an online stage, it is applied to unseen data to provide near-instantaneous results.

FIG. 11 shows a workflow 1100 for training and applying a machine-learning based model for predicting blood flow and contrast agent circulation parameters of a patient, in accordance with one or more embodiments. In one embodiment, the trained model resulting from workflow 1100 is the computational model generated at step 206 of FIG. 2. The trained machine-learning model resulting from workflow 1100 is trained to provide blood flow and contrast agent circulation parameters, such as, e.g., flow, velocities, contrast agent concentration, etc. from patient-specific input data. The blood flow and contrast agent circulation parameters determined by the trained machine-learning model can thus be used to determine administration parameters for administering a contrast agent and a trigger time for imaging the patient in steps 208 and 210 of FIG. 2. Blocks 1102-1110 show an offline or training stage for training a machine learning model and blocks 1112-1116 show an online stage for applying the trained machine learning model.

During an offline training stage, at step 1102, synthetic input training data is generated. The synthetic input training data may be generated by simulating a medical imaging system and stored in a database. The synthetic input training data may be synthetically generated geometries that are not based on patient specific data. Such geometries may be generated by varying the shape, severity, location, and number of stenoses, together with the radius and locations of main and side branches in a generic model of an artery. As a simplest example of a synthetically generated geometry, one can use a straight tube with a narrowing to represent the stenosis. Multiple CFD simulations can be performed by varying the synthetic geometry (e.g., minimum radius of the stenosis, entrance angle, exit angle) and varying the inflow or outflow boundary conditions to compute the FFR value. One advantage of using synthetically generated geometries is that it does not require the collection and processing of patient-specific data for completing the training phase, thereby saving both time and cost. Further, there is no limit on the type of synthetic geometries that can be generated, thereby covering a wide spectrum of vessel shapes and topology. Using this approach, the entire training phase can be performed without any patient-specific geometry or image data. United States Published Patent Application Nos. 20150112182 and 2014/0024932, which are incorporated herein by reference in their entirety, further describe synthetically generated geometries. In some embodiments, additional input training data (e.g., actual data of one or more patients or data generated from in vitro experiments) may additionally or alternatively be acquired at step 1102.

The input training data may include any suitable data for training a machine learning model to predict measures of interest. For example, the input training data may include patient characteristics, such as, e.g., arterial models, heart rate, blood pressure, weight, height, body surface area, age, gender, etc. The input training data may also include administration parameters for administering the contrast agent, such as, e.g., the volume of the contrast agent to be injected into the patient, the concentration of the contrast agent to be injected into the patient, and the rate and profile of the injection of the contrast agent into the patient.

At step 1104, blood flow and contrast agent circulation simulations are performed using the input data. In an exemplary embodiment, full body simulations are performed using the full body model described with respect to FIG. 5.

At step 1106, measures of interest are determined from the blood flow and contrast agent circulation calculations. The measures of interest may include any blood flow and contrast agent circulation parameter, such as, e.g., flows, velocities, concentrations of the contrast agent, etc. At step 1108, features are extracted from the input training data. The features are determined based on the characteristics available (e.g., in the database) for the input training data. It should be understood that step 1108 may be performed at any time prior to step 1110. At step 1110, a data-drive machine learning model is trained to predict the measures of interest based on the extracted features. The machine learning approaches may include, e.g., regression, instance-based methods, regularization methods, decision tree learning, Bayesian, kernel methods, clustering methods, association rule learning, artificial neural networks, dimensionality reduction, ensemble methods, or any other suitable machine learning approach. In one embodiment, the machine learning models may be trained using methods known in the art.

During an online stage, at step 1112, input data is received. The input data received at this step represents unseen data specific to the patient to be imaged. The input data may be any suitable data for predicting the measures of interest by the trained machine learning model. For example, the input data at step 1112 may include patient characteristics and administration parameters for a patient. The patient characteristics may include a patient-specific anatomical model. The patient-specific anatomical model may be generated from (e.g., previously generated) medical imaging data of the patient, a surface model of the patient, or physiological qualities of the patient, as discussed above with respect to the CFD model shown in FIG. 5. In one embodiment, the anatomical model may be generated from a surface model of a patient acquired as discussed above with respect to FIG. 8. The patient characteristics may also include, e.g., heart rate, blood pressure, weight, height, body surface area, age, gender, etc. The administration parameters may include, e.g., the volume of the contrast agent to be injected into the patient, the concentration of the contrast agent to be injected into the patient, and the rate and profile of the injection of the contrast agent into the patient.

At step 1114, features are extracted from the input data received at step 1112. At step 1116, measures of interest are predicted from the extracted features (extracted at step 1114) using the trained machine learning model (trained at step 1110). The measures of interest may include any blood flow and contrast agent circulation parameter. Advantageously, the trained machine-learning model can provide near instantaneous results for determining blood flow and contrast agent circulation parameters as the measures of interest, which may be used to determine administration characteristics for administering a contrast agent and a trigger time for imaging the patent. In one embodiment, separate machine learning algorithms may be trained and applied to provide a confidence interval for the estimation of the administration parameters.

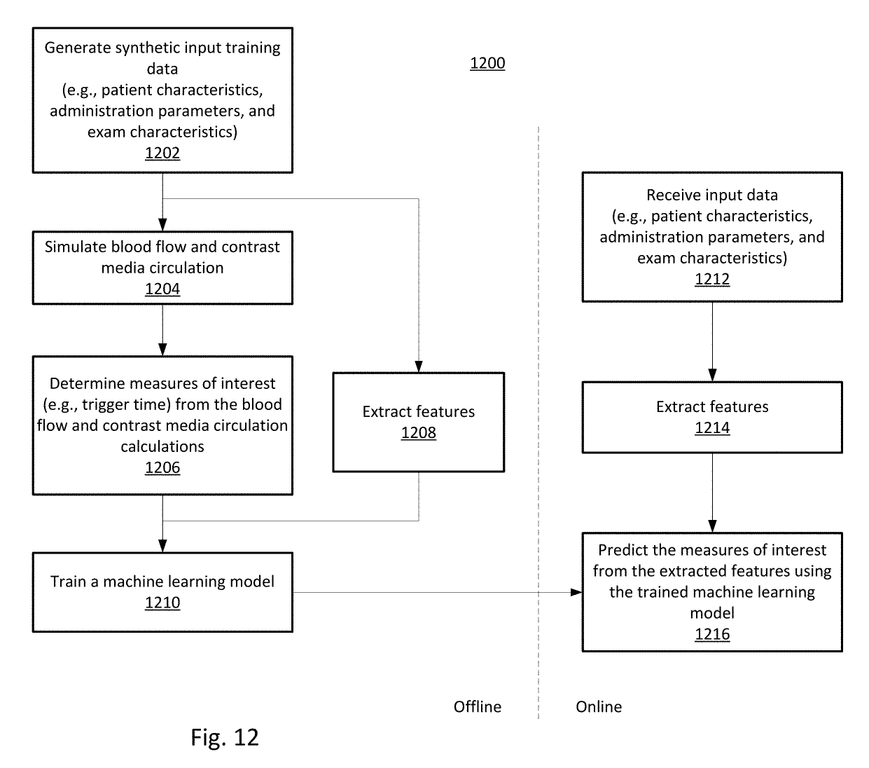

FIG. 12 shows a workflow 1200 for training and applying a machine-learning based model for predicting a trigger time to initiate imaging of a patient, in accordance with one or more embodiments. In one embodiment, the trained model resulting from workflow 1200 is the computational model generated at step 206 of FIG. 2. Blocks 1202-1210 show an offline or training stage for training a machine learning model and blocks 1212-1216 show an online stage for applying the trained machine learning model.

During an offline training stage, at step 1202, synthetic input training data is generated. The synthetic input training data may be generated by simulating a medical imaging system and stored in a database, as described above with respect to FIG. 11. The input training data may include any suitable data for training a machine learning model to predict measures of interest. For example, the input training data may include patient characteristics, such as, e.g., arterial models, heart rate, blood pressure, weight, height, body surface area, age, gender, etc. The input training data may also include administration parameters for administering the contrast agent, such as, e.g., the volume of the contrast agent to be injected into the patient, the concentration of the contrast agent to be injected into the patient, and the rate and profile of the injection of the contrast agent into the patient. The input training data may further include exam characteristics, such as, e.g., the region of interest to be imaged and the diagnostic/clinical issue.

At step 1204, blood flow and contrast agent circulation simulations are performed using the input data. In an exemplary embodiment, full body simulations are performed using the full body model described with respect to FIG. 5.

At step 1206, measures of interest are determined from the blood flow and contrast agent circulation calculations. The measures of interest may include the trigger time for initiating imaging of the patient (e.g., the bolus arrival time). At step 1208, features are extracted from the input training data. The features are determined based on the characteristics available (e.g., in the database) for the input training data. It should be understood that step 1208 may be performed at any time prior to step 1210. At step 1210, a data-drive machine learning model is trained to predict the measures of interest based on the extracted features. The machine learning approaches may include, e.g., regression, instance-based methods, regularization methods, decision tree learning, Bayesian, kernel methods, clustering methods, association rule learning, artificial neural networks, dimensionality reduction, ensemble methods, or any other suitable machine learning approach. In one embodiment, the machine learning models may be trained using methods known in the art.

During an online stage, at step 1212, input data is received. The input data received at this step represents unseen data specific to the patient to be imaged. The input data may be any suitable data for predicting the measures of interest by the trained machine learning model. For example, the input data at step 1112 may include patient characteristics, administration parameters for a patient, and exam characteristics. The patient characteristics may include a patient-specific anatomical model. The patient-specific anatomical model may be generated from (e.g., previously generated) medical imaging data of the patient, a surface model of the patient, or physiological qualities of the patient, as discussed above with respect to the CFD model shown in FIG. 5. In one embodiment, the anatomical model may be generated from a surface model of a patient acquired as discussed above with respect to FIG. 8. The patient characteristics may also include, e.g., heart rate, blood pressure, weight, height, body surface area, age, gender, etc. The administration parameters may include, e.g., the volume of the contrast agent to be injected into the patient, the concentration of the contrast agent to be injected into the patient, and the rate and profile of the injection of the contrast agent into the patient. The exam characteristics may include, e.g., the region of interest to be imaged and the diagnostic/clinical issue.

At step 1214, features are extracted from the input data received at step 1212. At step 1216, measures of interest are predicted from the extracted features (extracted at step 1214) using the trained machine learning model (trained at step 1210). The measures of interest may include the trigger time for initiating imaging of a patient. Advantageously, the trained machine-learning model can provide near instantaneous results for determining the trigger time as the measures of interest. In one embodiment, separate machine learning algorithms may be trained and applied to provide a confidence interval for the estimation of the administration parameters.

In one embodiment, the trained machine-learning model described with respect to FIG. 11 may be the computational model applied at step 208 of FIG. 2 and the trained machine-learning model described with respect to FIG. 12 may be the computational model applied at step 210 of FIG. 2.

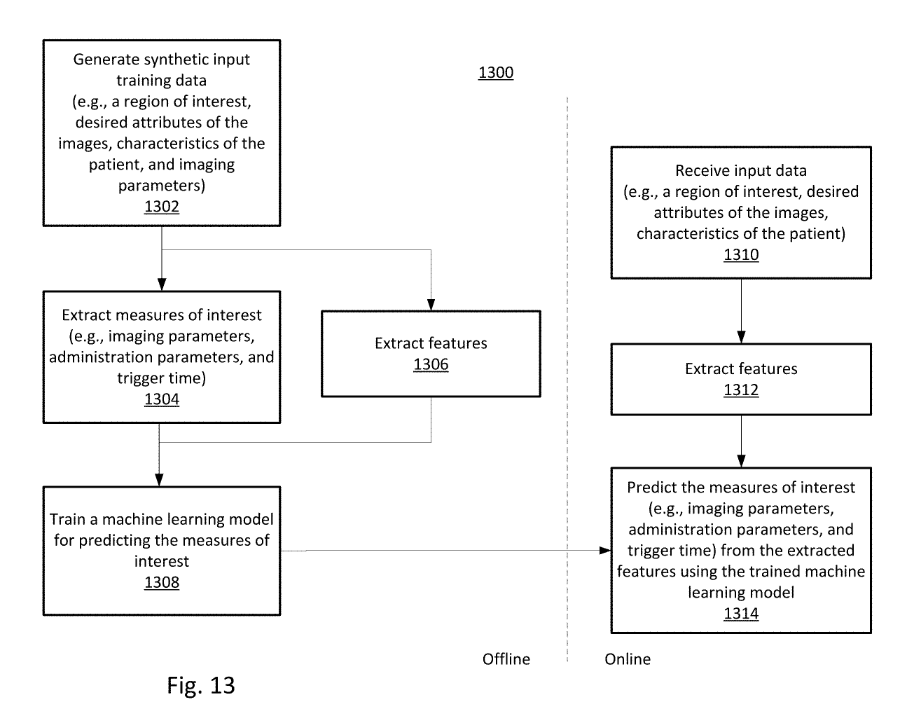

In one embodiment, a machine learning model may be learned to determine the imaging parameters, the administration parameters, and the trigger time. FIG. 13 shows a workflow 1300 for training and applying a machine learning model for determining the imaging parameters and the administration parameters, in accordance with one or more embodiments. The trained machine learning model resulting from workflow 1300 is trained to determine imaging parameters for imaging a patient. In one embodiment, the trained machine learning model resulting from workflow 1300 replaces method 200 of FIG. 2. Blocks 1302-1308 show an offline or training stage for training a machine learning model and blocks 1310-1314 show an online stage for applying the trained machine learning model.

During the offline stage, at step 1302, synthetic input training data is generated. The synthetic input training data may be generated as described above with respect to FIG. 11. In some embodiments, additional input training data (e.g., actual data of one or more patients or data generated from in vitro experiments) may additionally or alternatively be acquired at step 1302. The input training data may be any suitable data for training a machine learning model to predict measures of interest. For example, the input training data may include a region of interest, desired attributes of the images to be generated for the region of interest, characteristics of a patient, imaging parameters for imaging the region of interest, and administration parameters for administering the contrast agent.

At step 1304, measures of interest are extracted from the input training data. In this embodiment, the measures of interest include imaging parameters for imaging the region of interest, such as, e.g., parameters of the contrast agent (e.g., the concentration of the contrast agent in the region of interest), parameters of the medical imaging system (e.g., tube voltage, tube current, exposure time (i.e., duration of scan), table speed, the reconstruction algorithm (e.g., filter, convolution kernel properties, etc.), and medical imaging system specifications (e.g., beam spectra, geometry, x-ray beam collimation, etc.). The measures of interest may also include administration parameters for administering the contrast agent (e.g., the volume of the contrast agent to be injected into the patient, the concentration of the contrast agent to be injected into the patient, and the rate and profile of the injection of the contrast agent into the patient). The measures of interest may further include the trigger time for initiating imaging of the patient.

At step 1306, features are extracted from the input training data. The features are determined based on the characteristics available (e.g., in the database) for the input training data. It should be understood that step 1306 may be performed at any time prior to step 1308. At step 1308, one or more machine learning models are trained to predict the measures of interest based on the extracted features. The machine learning approaches may include, e.g., regression, instance-based methods, regularization methods, decision tree learning, Bayesian, kernel methods, clustering methods, association rule learning, artificial neural networks, dimensionality reduction, ensemble methods, or any other suitable machine learning approach. In one embodiment, the machine learning models may be trained using methods known in the art. In one embodiment, the machine learning models may be trained using methods known in the art.

During the online stage, at step 1310, input data is received. The input data received at this step represents unseen data of the patient to be imaged. The input data may be any suitable data for predicting the measures of interest by the trained machine learning model. For example, the input data at step 1310 may include a region of interest of the patient, desired attributes of the images to be generated for the region of interest of the patient, and characteristics of the patient. At step 1312, features are extracted from the input data. At step 1314, the measures of interest are predicted from the extracted features using the trained machine learning model. The measures of interest may include imaging parameters for imaging the region of interest and administration parameters for administering the contrast agent.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps of the methods and workflows described herein, including one or more of the steps of FIGS. 2, 4, and 8-13. Certain steps of the methods and workflows described herein, including one or more of the steps of FIGS. 2, 4, and 8-13, may be performed by a server or by another processor in a network-based cloud-computing system. Certain steps of the methods and workflows described herein, including one or more of the steps of FIGS. 2, 4, and 8-13, may be performed by a client computer in a network-based cloud computing system. The steps of the methods and workflows described herein, including one or more of the steps of FIGS. 2, 4, and 8-13, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps of FIGS. 2, 4, and 8-13, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

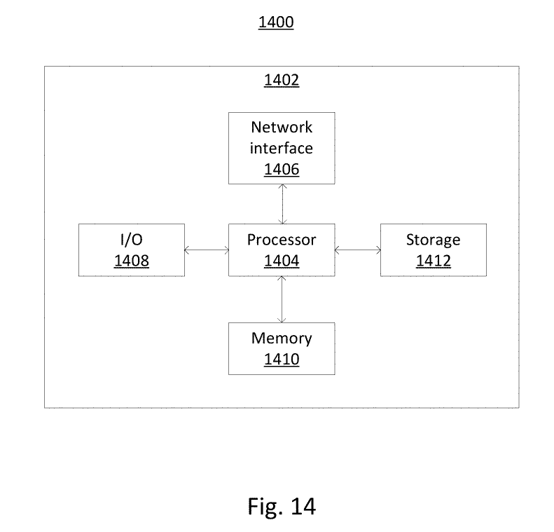

A high-level block diagram 1400 of an example computer that may be used to implement systems, apparatus, and methods described herein is depicted in FIG. 14. Computer 1402 includes a processor 1404 operatively coupled to a data storage device 1412 and a memory 1410. Processor 1404 controls the overall operation of computer 1402 by executing computer program instructions that define such operations. The computer program instructions may be stored in data storage device 1412, or other computer readable medium, and loaded into memory 1410 when execution of the computer program instructions is desired. Thus, the method and workflow steps of FIGS. 2, 4, and 8-13 can be defined by the computer program instructions stored in memory 1410 and/or data storage device 1412 and controlled by processor 1404 executing the computer program instructions. For example, the computer program instructions can be implemented as computer executable code programmed by one skilled in the art to perform the method and workflow steps of FIGS. 2, 4, and 8-13. Accordingly, by executing the computer program instructions, the processor 1404 executes the method and workflow steps of FIGS. 2, 4, and 8-13. Computer 1404 may also include one or more network interfaces 1406 for communicating with other devices via a network. Computer 1402 may also include one or more input/output devices 1408 that enable user interaction with computer 1402 (e.g., display, keyboard, mouse, speakers, buttons, etc.).

Processor 1404 may include both general and special purpose microprocessors, and may be the sole processor or one of multiple processors of computer 1402. Processor 1404 may include one or more central processing units (CPUs), for example. Processor 1404, data storage device 1412, and/or memory 1410 may include, be supplemented by, or incorporated in, one or more application-specific integrated circuits (ASICs) and/or one or more field programmable gate arrays (FPGAs).

Data storage device 1412 and memory 1410 each include a tangible non-transitory computer readable storage medium. Data storage device 1412, and memory 1410, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1408 may include peripherals, such as a printer, scanner, display screen, etc. For example, input/output devices 1408 may include a display device such as a cathode ray tube (CRT) or liquid crystal display (LCD) monitor for displaying information to the user, a keyboard, and a pointing device such as a mouse or a trackball by which the user can provide input to computer 1402.

Any or all of the systems and apparatus discussed herein, including elements of workstation 102 of FIG. 1, may be implemented using one or more computers such as computer 1402.

One skilled in the art will recognize that an implementation of an actual computer or computer system may have other structures and may contain other components as well, and that FIG. 14 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A method for imaging a patient, comprising:
determining target imaging parameters for imaging a region of interest of a patient based on desired attributes of images to be generated for the region of interest;
determining administration parameters for administering a contrast agent based on the target imaging parameters using a computational model of blood flow and contrast agent circulation;
determining a trigger time for imaging the region of interest based on the administration parameters using the computational model of blood flow and contrast agent circulation; and
causing the region of interest of the patient to be imaged based on the administration parameters and the trigger time.

2. The method of claim 1, wherein the computational model is a computational fluid dynamics model personalized to model blood flow and contrast agent circulation of the patient.

3. The method of claim 2, further comprising:
personalizing the computational fluid dynamics model based on medical imaging data of the patient.

4. The method of claim 2, further comprising:
personalizing the computational fluid dynamics model based on an anatomical model, the anatomical model generated by:
acquiring three-dimensional surface imaging data of the patient;
generating a surface model of the patient from the three-dimensional surface imaging data; and
matching the surface model of the patient to the anatomical model.

5. The method of claim 1, wherein determining administration parameters for administering a contrast agent based on the target imaging parameters using a computational model of blood flow and contrast agent circulation comprises:
determining the administration parameters that result in a minimum error between the target imaging parameters and computed imaging parameters, the computed imaging parameters computed from the computation model of blood flow and contrast agent circulation using the administration parameters.

6. The method of claim 1, wherein determining a trigger time for imaging the region of interest based on the administration parameters using the computational model of blood flow and contrast agent circulation comprises:
determining concentration levels of the contrast agent in the region of interest using the computational model of blood flow and contrast agent circulation and the administration parameters; and
determining a time that the concentration levels is at its maximum as the trigger time.

7. The method of claim 1, wherein the computational model is a machine learning based model trained to predict blood flow and contrast agent circulation of the patient.

8. The method of claim 7, further comprising training the machine learning based model by:
generating synthetic training data comprising patient characteristics and administration parameters;
simulating blood flow and contrast media circulation for the synthetic training data using a computational fluid dynamics model;
determining blood flow and contrast media circulation parameters for the synthetic training data from the simulating;
extracting features from the synthetic training data; and
training the machine learning based model to predict the blood flow and contrast media circulation parameters using the features extracted from the synthetic training data.

9. The method of claim 8, further comprising:
generating a patient-specific anatomical model specific to the patient by:
acquiring three-dimensional surface imaging data of the patient;
generating a surface model of the patient from the three-dimensional surface imaging data; and
matching the surface model of the patient to an anatomical model to provide the patient-specific anatomical model;
extracting features from the generated patient-specific anatomical model; and
predicting the blood flow and contrast media circulation parameters based on the features extracted from the generated patient-specific anatomical model, wherein the administration parameters and the trigger time are determined based on the predicted blood flow and contrast media circulation parameters.

10. The method of claim 7, further comprising training the machine learning based model by:
generating synthetic training data comprising patient characteristics, administration parameters, and regions of interest;
simulating blood flow and contrast media circulation for the synthetic training data using a computational fluid dynamics model;
determining times for imaging the regions of interest from the simulating;
extracting features from the synthetic training data; and
training the machine learning based model to predict the times for imaging the regions of interest using the extracted features.

11. The method of claim 1, wherein causing the region of interest of the patient to be imaged based on the administration parameters and the trigger time comprises at least one of:
automatically initiating imaging of the region of interest of the patient at the trigger time; or
notifying a user to manually initiate imaging of the region of interest of the patient at the trigger time.

12. An apparatus for imaging a patient, comprising:
means for determining target imaging parameters for imaging a region of interest of a patient based on desired attributes of images to be generated for the region of interest;
means for determining administration parameters for administering a contrast agent based on the target imaging parameters using a computational model of blood flow and contrast agent circulation;
means for determining a trigger time for imaging the region of interest based on the administration parameters using the computational model of blood flow and contrast agent circulation; and
means for causing the region of interest of the patient to be imaged based on the administration parameters and the trigger time.

13. The apparatus of claim 12, wherein the computational model is a computational fluid dynamics model personalized to model blood flow and contrast agent circulation of the patient.

14. The apparatus of claim 13, further comprising:
means for personalizing the computational fluid dynamics model based on medical imaging data of the patient.

15. The apparatus of claim 13, further comprising:
means for personalizing the computational fluid dynamics model based on an anatomical model, the anatomical model generated by:
means for acquiring three-dimensional surface imaging data of the patient;
means for generating a surface model of the patient from the three-dimensional surface imaging data; and
means for matching the surface model of the patient to the anatomical model.

16. The apparatus of claim 12, wherein determining administration parameters for administering a contrast agent based on the target imaging parameters using a computational model of blood flow and contrast agent circulation comprises:
means for determining the administration parameters that result in a minimum error between the target imaging parameters and computed imaging parameters, the computed imaging parameters computed from the computation model of blood flow and contrast agent circulation using the administration parameters.

17. The apparatus of claim 12, wherein determining a trigger time for imaging the region of interest based on the administration parameters using the computational model of blood flow and contrast agent circulation comprises:
means for determining concentration levels of the contrast agent in the region of interest using the computational model of blood flow and contrast agent circulation and the administration parameters; and
means for determining a time that the concentration levels is at its maximum as the trigger time.

18. The apparatus of claim 12, wherein the computational model is a machine learning based model trained to predict blood flow and contrast agent circulation of the patient.

19. A non-transitory computer readable medium storing computer program instructions for imaging a patient, the computer program instructions when executed by a processor cause the processor to perform operations comprising:
determining target imaging parameters for imaging a region of interest of a patient based on desired attributes of images to be generated for the region of interest;
determining administration parameters for administering a contrast agent based on the target imaging parameters using a computational model of blood flow and contrast agent circulation;
determining a trigger time for imaging the region of interest based on the administration parameters using the computational model of blood flow and contrast agent circulation; and
causing the region of interest of the patient to be imaged based on the administration parameters and the trigger time.

20. The non-transitory computer readable medium of claim 19, wherein the computational model is a computational fluid dynamics model personalized to model blood flow and contrast agent circulation of the patient.

21. The non-transitory computer readable medium of claim 19, wherein the computational model is a machine learning based model trained to predict blood flow and contrast agent circulation of the patient.

22. The non-transitory computer readable medium of claim 21, further comprising training the machine learning based model by:
generating synthetic training data comprising patient characteristics and administration parameters;
simulating blood flow and contrast media circulation for the synthetic training data using a computational fluid dynamics model;
determining blood flow and contrast media circulation parameters for the synthetic training data from the simulating;
extracting features from the synthetic training data; and
training the machine learning based model to predict the blood flow and contrast media circulation parameters using the features extracted from the synthetic training data.

23. The non-transitory computer readable medium of claim 22, further comprising:
generating a patient-specific anatomical model specific to the patient by:
acquiring three-dimensional surface imaging data of the patient;
generating a surface model of the patient from the three-dimensional surface imaging data; and
matching the surface model of the patient to an anatomical model to provide the patient-specific anatomical model;
extracting features from the generated patient-specific anatomical model; and
predicting the blood flow and contrast media circulation parameters based on the features extracted from the generated patient-specific anatomical model, wherein the administration parameters and the trigger time are determined based on the predicted blood flow and contrast media circulation parameters.

24. The non-transitory computer readable medium of claim 22, further comprising training the machine learning based model by:
generating synthetic training data comprising patient characteristics, administration parameters, and regions of interest;
simulating blood flow and contrast media circulation for the synthetic training data using a computational fluid dynamics model;
determining times for imaging the regions of interest from the simulating;
extracting features from the synthetic training data; and
training the machine learning based model to predict the times for imaging the regions of interest using the extracted features.

25. The non-transitory computer readable medium of claim 19, wherein causing the region of interest of the patient to be imaged based on the administration parameters and the trigger time comprises at least one of:
automatically initiating imaging of the region of interest of the patient at the trigger time; or
notifying a user to manually initiate imaging of the region of interest of the patient at the trigger time.
